(19) United States
(12) Patent Application Publication
(10) Pub. No.: US 2020/0245928 A1
(43) Pub. Date: Aug. 6, 2020

(54) METHOD FOR MANAGING WEIGHT OF USER AND ELECTRONIC DEVICE THEREFOR

(71) Applicant: Samsung Electronics Co., Ltd., Gyeonggi-do (KR)

(72) Inventors: Ki-Man KANG, Gyeonggi-do (KR); Hyeyoon KANG, Gyeonggi-do (KR); Woong Jo WOO, Seoul (KR)

(21) Appl. No.: 16/642,448

(22) PCT Filed: Aug. 28, 2018
(86) PCT No.: PCT/KR2018/009920

§ 371 Date: Feb. 27, 2020

(30) Foreign Application Priority Data
Aug. 31, 2017 (KR) 10-2017-0110898

(51) Int. Cl. A61B 5/00; H04M 1/725; G06F 3/0488; G16H 20/30; G16H 40/67; G16H 20/60; A61B 5/11

(52) U.S. Cl. CPC A61B 5/4866; H04M 1/72569; G06F 3/0488; G16H 20/30; G16H 40/67; G06F 3/04847; A61B 5/1123; A61B 5/6898; A61B 5/486; H04M 2250/12; G16H 20/60

(57) ABSTRACT

Various embodiments of the present invention relate to a device and a method for managing the weight of a user in an electronic device. The electronic device comprises: a housing; a touch screen display exposed through at least a part of the housing; a motion sensor disposed in the housing; a wireless communication circuit disposed in the housing; a processor electrically connected to the touch screen display, the motion sensor and the wireless communication circuit, and disposed in the housing; and a memory electrically connected to the processor and disposed in the housing, wherein the memory can store instructions, during the execution thereof, allowing the processor to: use the motion sensor so as to obtain data related to a movement of the user carrying the electronic device; determine the type and the duration of the movement on the basis of at least a part of the obtained data; obtain a first value related to the food intake of the user; determine a second value indicating the calories consumed by the user on the basis of at least a part of a weighted value related to the type of movement and the type and the duration of the movement; determine a third value on the basis of at least a part of the first value and the second value; and provide a graphic user interface to the touch screen display on the basis of at least a part of the third value. Other embodiments are also possible.

### TECHNICAL FIELD

Various embodiments of the disclosure relates to a device and a method for managing a user's weight in an electronic device.

### BACKGROUND ART

With the development of information communication technology and semiconductor technology, various types of electronic devices have been developed to provide various functions. For example, the various functions may include at least one among a voice communication function, a message function, a camera function, an electronic payment function, an Internet function, or a music reproduction function.

An electronic device may provide a health-care function in order to reflect users' interest in health care. For example, an electronic device may provide a user with information on exercise, such as rope skipping, walking, or muscular movement, through an exercise application.

### DISCLOSURE OF INVENTION

### Technical Problem

An electronic device may provide a health-care function to measure the amount of exercise by a user corresponding to a specific exercise (e.g. walking, cycling, etc.). For example, the electronic device may measure the number of a user's steps or how far and how long the user continuously walks for a day.

With increased interest in health care, a user of the electronic device requires various types of information on a change in the user's body in addition to the amount of exercise by the user. Therefore, the electronic device requires a method for providing various types of information related to the user's health.

Various embodiments of the disclosure may provide a device and a method for managing a user's weight in an electronic device.

### Solution to Problem

According to various embodiments of the disclosure, an electronic device may include: a housing; a touch-screen display exposed through at least a part of the housing; a motion sensor disposed in the housing; a wireless communication circuit disposed in the housing; a processor disposed in the housing and electrically connected to the touch-screen display, the motion sensor, and the wireless communication circuit; and a memory disposed in the housing and electrically connected to the processor, wherein the memory may store instructions which cause, when executed, the processor to: acquire, using the motion sensor, data related to motion of a user carrying the electronic device; determine a duration and a type of the motion, based at least partially on the acquired data; acquire a first value related to the user's food intake; determine a second value indicating the amount of calories burned by the user, based at least partially on the duration and the type of the motion and a weighted value related to the type of the motion; determine a third value, based at least partially on the first value and the second value; and provide, based at least partially on the third value, a graphical user interface to the touch-screen display.

According to various embodiments of the disclosure, an operation method of an electronic device may include: acquiring data related to motion of a user carrying the electronic device; determining a duration and a type of the motion, based at least partially on the acquired data; acquiring a first value related to the user's food intake; determining a second value indicating the amount of calories burned by the user, based at least partially on the duration and the type of the motion and a weighted value related to the type of the motion; determining a third value, based at least partially on the first value and the second value; and providing, based at least partially on the third value, a graphical user interface to a touch-screen display electrically connected to the electronic device.

### Advantageous Effects of Invention

The electronic device and the operation method thereof according to various embodiments can, when a weight management mode is configured based on information on a user (e.g. the user's profile, a use history of an electronic device, a user input, etc.), display user state information related to a target weight so that the user can easily recognize the user's body activity and exercise information necessary for weight management.

### BRIEF DESCRIPTION OF DRAWINGS

FIG. 1 illustrates a block diagram of an electronic device in a network environment for a user's weight management according to various embodiments of the disclosure;

FIG. 2 illustrates a flowchart for a user's weight management in an electronic device according to various embodiments of the disclosure;

FIGS. 3A to 3C illustrate a screen configuration of an electronic device for collecting data related to a user according to various embodiments of the disclosure;

FIG. 4 illustrates a flowchart for configuring a weight management mode in an electronic device according to various embodiments of the disclosure;

FIG. 5 illustrates a flowchart for configuring target calories in an electronic device according to various embodiments of the disclosure;

FIG. 6 illustrates a screen configuration of an electronic device for configuring target calories according to various embodiments of the disclosure;

FIG. 7 illustrates a flowchart for displaying a user's state information for weight management in an electronic device according to various embodiments of the disclosure;

FIGS. 8A to 8D illustrate a configuration of a user interface including a user's state information on a daily basis in an electronic device according to various embodiments of the disclosure;

FIGS. 9A to 9F illustrate a configuration of a user interface including a user's state information on a monthly basis in an electronic device according to various embodiments of the disclosure;

FIG. 10 illustrates a flowchart for identifying a calorie intake amount in an electronic device according to various embodiments of the disclosure;

FIG. 11 illustrates a flowchart for displaying exercise guidance information in an electronic device according to various embodiments of the disclosure;

FIG. 12 illustrates a flowchart for reconfiguring a target weight amount in an electronic device according to various embodiments of the disclosure; and

FIGS. 13A to 13C illustrate a screen configuration of an electronic device corresponding to achieving a weight management goal according to various embodiments of the disclosure.

### BEST MODE FOR CARRYING OUT THE INVENTION

Hereinafter, various embodiments of the disclosure will be described in detail with reference to the accompanying drawings. Further, in the following description of embodiments of the disclosure, a detailed description of known functions and configurations incorporated herein will be omitted when it is considered to make the subject matter of the disclosure rather unclear. The terms used below are terms defined in consideration of the functions in the disclosure, and may differ according to users, intentions of users, or customs. Therefore, the definitions of the terms should be made based on the contents throughout the specification.

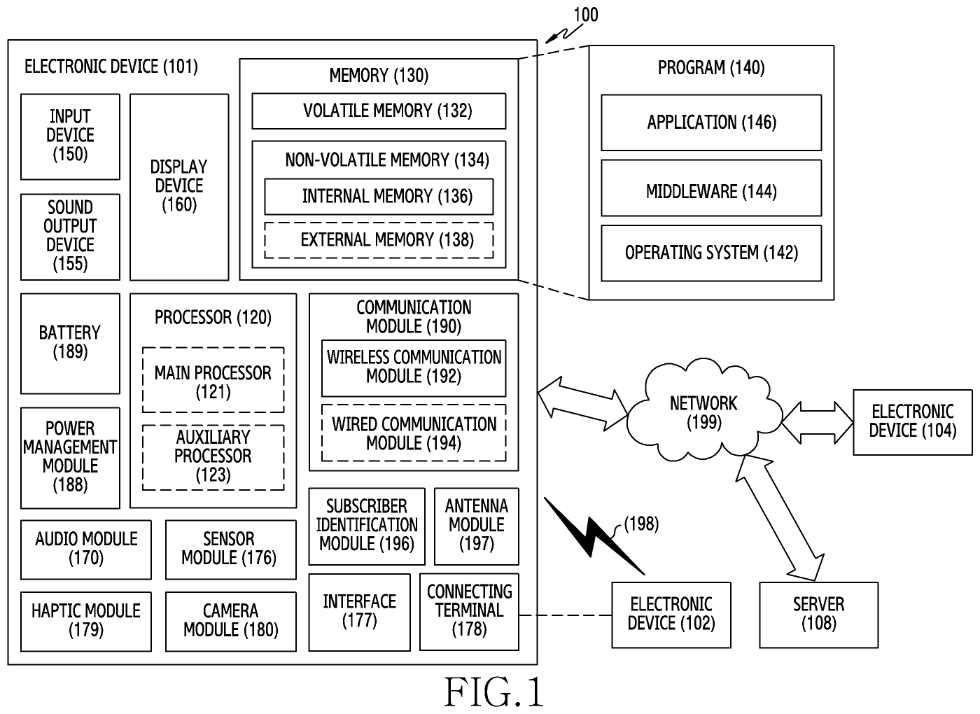

FIG. 1 is a block diagram illustrating an electronic device 101 in a network environment 100 according to various embodiments. Referring to FIG. 1, the electronic device 101 in the network environment 100 may communicate with an electronic device 102 via a first network 198 (e.g., a short-range wireless communication network), or an electronic device 104 or a server 108 via a second network 199 (e.g., a long-range wireless communication network). According to an embodiment, the electronic device 101 may communicate with the electronic device 104 via the server 108. According to an embodiment, the electronic device 101 may include a processor 120, memory 130, an input device 150, a sound output device 155, a display device 160, an audio module 170, a sensor module 176, an interface 177, a haptic module 179, a camera module 180, a power management module 188, a battery 189, a communication module 190, a subscriber identification module (SIM) 196, or an antenna module 197. In some embodiments, at least one (e.g., the display device 160 or the camera module 180) of the components may be omitted from the electronic device 101, or one or more other components may be added in the electronic device 101. In some embodiments, some of the components may be implemented as single integrated circuitry. For example, the sensor module 176 (e.g., a fingerprint sensor, an iris sensor, or an illuminance sensor) may be implemented as embedded in the display device 160 (e.g., a display).

The processor 120 may execute, for example, software (e.g., a program 140) to control at least one other component (e.g., a hardware or software component) of the electronic device 101 coupled with the processor 120, and may perform various data processing or computation. According to one embodiment, as at least part of the data processing or computation, the processor 120 may load a command or data received from another component (e.g., the sensor module 176 or the communication module 190) in volatile memory 132, process the command or the data stored in the volatile memory 132, and store resulting data in non-volatile memory 134. According to an embodiment, the processor 120 may include a main processor 121 (e.g., a central processing unit (CPU) or an application processor (AP)), and an auxiliary processor 123 (e.g., a graphics processing unit (GPU), an image signal processor (ISP), a sensor hub processor, or a communication processor (CP)) that is operable independently from, or in conjunction with, the main processor 121. Additionally or alternatively, the auxiliary processor 123 may be adapted to consume less power than the main processor 121, or to be specific to a specified function. The auxiliary processor 123 may be implemented as separate from, or as part of the main processor 121.

The auxiliary processor 123 may control at least some of functions or states related to at least one component (e.g., the display device 160, the sensor module 176, or the communication module 190) among the components of the electronic device 101, instead of the main processor 121 while the main processor 121 is in an inactive (e.g., sleep) state, or together with the main processor 121 while the main processor 121 is in an active state (e.g., executing an application). According to an embodiment, the auxiliary processor 123 (e.g., an image signal processor or a communication processor) may be implemented as part of another component (e.g., the camera module 180 or the communication module 190) functionally related to the auxiliary processor 123. The memory 130 may store various data used by at least one component (e.g., the processor 120 or the sensor module 176) of the electronic device 101. The various data may include, for example, software (e.g., the program 140) and input data or output data for a command related thereto. The memory 130 may include the volatile memory 132 or the non-volatile memory 134.

The program 140 may be stored in the memory 130 as software, and may include, for example, an operating system (OS) 142, middleware 144, or an application 146.

The input device 150 may receive a command or data to be used by other component (e.g., the processor 120) of the electronic device 101, from the outside (e.g., a user) of the electronic device 101. The input device 150 may include, for example, a microphone, a mouse, or a keyboard.

The sound output device 155 may output sound signals to the outside of the electronic device 101. The sound output device 155 may include, for example, a speaker or a receiver. The speaker may be used for general purposes, such as playing multimedia or playing record, and the receiver may be used for an incoming calls. According to an embodiment, the receiver may be implemented as separate from, or as part of the speaker.

The display device 160 may visually provide information to the outside (e.g., a user) of the electronic device 101. The display device 160 may include, for example, a display, a hologram device, or a projector and control circuitry to control a corresponding one of the display, hologram device, and projector. According to an embodiment, the display device 160 may include touch circuitry adapted to detect a touch, or sensor circuitry (e.g., a pressure sensor) adapted to measure the intensity of force incurred by the touch.

The audio module 170 may convert a sound into an electrical signal and vice versa. According to an embodiment, the audio module 170 may obtain the sound via the input device 150, or output the sound via the sound output device 155 or a headphone of an external electronic device (e.g., an electronic device 102) directly (e.g., wired) or wirelessly coupled with the electronic device 101.

The sensor module 176 may detect an operational state (e.g., power or temperature) of the electronic device 101 or an environmental state (e.g., a state of a user) external to the electronic device 101, and then generate an electrical signal or data value corresponding to the detected state. According to an embodiment, the sensor module 176 may include, for example, a gesture sensor, a gyro sensor, an atmospheric pressure sensor, a magnetic sensor, an acceleration sensor, a grip sensor, a proximity sensor, a color sensor, an infrared (IR) sensor, a biometric sensor, a temperature sensor, a humidity sensor, or an illuminance sensor.

The interface 177 may support one or more specified protocols to be used for the electronic device 101 to be coupled with the external electronic device (e.g., the electronic device 102) directly (e.g., wired) or wirelessly. According to an embodiment, the interface 177 may include, for example, a high definition multimedia interface (HDMI), a universal serial bus (USB) interface, a secure digital (SD) card interface, or an audio interface.

A connecting terminal 178 may include a connector via which the electronic device 101 may be physically connected with the external electronic device (e.g., the electronic device 102). According to an embodiment, the connecting terminal 178 may include, for example, a HDMI connector, a USB connector, a SD card connector, or an audio connector (e.g., a headphone connector),

The haptic module 179 may convert an electrical signal into a mechanical stimulus (e.g., a vibration or a movement) or electrical stimulus which may be recognized by a user via his tactile sensation or kinesthetic sensation. According to an embodiment, the haptic module 179 may include, for example, a motor, a piezoelectric element, or an electric stimulator.

The camera module 180 may capture a still image or moving images. According to an embodiment, the camera module 180 may include one or more lenses, image sensors, image signal processors, or flashes.

The power management module 188 may manage power supplied to the electronic device 101. According to one embodiment, the power management module 188 may be implemented as at least part of, for example, a power management integrated circuit (PMIC).

The battery 189 may supply power to at least one component of the electronic device 101. According to an embodiment, the battery 189 may include, for example, a primary cell which is not rechargeable, a secondary cell which is rechargeable, or a fuel cell.

The communication module 190 may support establishing a direct (e.g., wired) communication channel or a wireless communication channel between the electronic device 101 and the external electronic device (e.g., the electronic device 102, the electronic device 104, or the server 108) and performing communication via the established communication channel. The communication module 190 may include one or more communication processors that are operable independently from the processor 120 (e.g., the application processor (AP)) and supports a direct (e.g., wired) communication or a wireless communication. According to an embodiment, the communication module 190 may include a wireless communication module 192 (e.g., a cellular communication module, a short-range wireless communication module, or a global navigation satellite system (GNSS) communication module) or a wired communication module 194 (e.g., a local area network (LAN) communication module or a power line communication (PLC) module). A corresponding one of these communication modules may communicate with the external electronic device via the first network 198 (e.g., a short-range communication network, such as BLUETOOTH, wireless-fidelity (Wi-Fi) direct, or infrared data association (IrDA)) or the second network 199 (e.g., a long-range communication network, such as a cellular network, the Internet, or a computer network (e.g., LAN or wide area network (WAN)). These various types of communication modules may be implemented as a single component (e.g., a single chip), or may be implemented as multi components (e.g., multi chips) separate from each other.

The wireless communication module 192 may identify and authenticate the electronic device 101 in a communication network, such as the first network 198 or the second network 199, using subscriber information (e.g., international mobile subscriber identity (IMSI)) stored in the subscriber identification module 196.

The antenna module 197 may transmit or receive a signal or power to or from the outside (e.g., the external electronic device) of the electronic device 101. According to an embodiment, the antenna module 197 may include one or more antennas, and, therefrom, at least one antenna appropriate for a communication scheme used in the communication network, such as the first network 198 or the second network 199, may be selected, for example, by the communication module 190 (e.g., the wireless communication module 192). The signal or the power may then be transmitted or received between the communication module 190 and the external electronic device via the selected at least one antenna.

At least some of the above-described components may be coupled mutually and communicate signals (e.g., commands or data) therebetween via an inter-peripheral communication scheme (e.g., a bus, general purpose input and output (GPIO), serial peripheral interface (SPI), or mobile industry processor interface (MIPI)).

According to an embodiment, commands or data may be transmitted or received between the electronic device 101 and the external electronic device 104 via the server 108 coupled with the second network 199. Each of the electronic devices 102 and 104 may be a device of a same type as, or a different type, from the electronic device 101. According to an embodiment, all or some of operations to be executed at the electronic device 101 may be executed at one or more of the external electronic devices 102, 104, or 108. For example, if the electronic device 101 should perform a function or a service automatically, or in response to a request from a user or another device, the electronic device 101, instead of, or in addition to, executing the function or the service, may request the one or more external electronic devices to perform at least part of the function or the service. The one or more external electronic devices receiving the request may perform the at least part of the function or the service requested, or an additional function or an additional service related to the request, and transfer an outcome of the performing to the electronic device 101. The electronic device 101 may provide the outcome, with or without further processing of the outcome, as at least part of a reply to the request. To that end, a cloud computing, distributed computing, or client-server computing technology may be used, for example.

The electronic device according to various embodiments may be one of various types of electronic devices. The electronic devices may include, for example, a portable communication device (e.g., a smart phone), a computer device, a portable multimedia device, a portable medical device, a camera, a wearable device, or a home appliance. According to an embodiment of the disclosure, the electronic devices are not limited to those described above.

It should be appreciated that various embodiments of the present disclosure and the terms used therein are not intended to limit the technological features set forth herein to particular embodiments and include various changes, equivalents, or replacements for a corresponding embodiment. With regard to the description of the drawings, similar reference numerals may be used to refer to similar or related elements. It is to be understood that a singular form of a noun corresponding to an item may include one or more of the things, unless the relevant context clearly indicates otherwise. As used herein, each of such phrases as “A or B,” “at least one of A and B,” “at least one of A or B,” “A, B, or C,” “at least one of A, B, and C,” and “at least one of A, B, or C,” may include all possible combinations of the items enumerated together in a corresponding one of the phrases. As used herein, such terms as “1st” and “2nd,” or “first” and “second” may be used to simply distinguish a corresponding component from another, and does not limit the components in other aspect (e.g., importance or order). It is to be understood that if an element (e.g., a first element) is referred to, with or without the term “operatively” or “communicatively”, as “coupled with,” “coupled to,” “connected with,” or “connected to” another element (e.g., a second element), it means that the element may be coupled with the other element directly (e.g., wired), wirelessly, or via a third element.

As used herein, the term “module” may include a unit implemented in hardware, software, or firmware, and may interchangeably be used with other terms, for example, “logic,” “logic block,” “part,” or “circuitry”. A module may be a single integral component, or a minimum unit or part thereof, adapted to perform one or more functions. For example, according to an embodiment, the module may be implemented in a form of an application-specific integrated circuit (ASIC).

Various embodiments as set forth herein may be implemented as software (e.g., the program 140) including one or more instructions that are stored in a storage medium (e.g., internal memory 136 or external memory 138) that is readable by a machine (e.g., the electronic device 101). For example, a processor (e.g., the processor 120) of the machine (e.g., the electronic device 101) may invoke at least one of the one or more instructions stored in the storage medium, and execute it, with or without using one or more other components under the control of the processor. This allows the machine to be operated to perform at least one function according to the at least one instruction invoked. The one or more instructions may include a code generated by a complier or a code executable by an interpreter. The machine-readable storage medium may be provided in the form of a non-transitory storage medium. Wherein, the term “non-transitory” simply means that the storage medium is a tangible device, and does not include a signal (e.g., an electromagnetic wave), but this term does not differentiate between where data is semi-permanently stored in the storage medium and where the data is temporarily stored in the storage medium.

According to an embodiment, a method according to various embodiments of the disclosure may be included and provided in a computer program product. The computer program product may be traded as a product between a seller and a buyer. The computer program product may be distributed in the form of a machine-readable storage medium (e.g., compact disc read only memory (CD-ROM)), or be distributed (e.g., downloaded or uploaded) online via an application store (e.g., PLAYSTORE), or between two user devices (e.g., smart phones) directly. If distributed online, at least part of the computer program product may be temporarily generated or at least temporarily stored in the machine-readable storage medium, such as memory of the manufacturer's server, a server of the application store, or a relay server.

According to various embodiments, each component (e.g., a module or a program) of the above-described components may include a single entity or multiple entities. According to various embodiments, one or more of the above-described components may be omitted, or one or more other components may be added. Alternatively or additionally, a plurality of components (e.g., modules or programs) may be integrated into a single component. In such a case, according to various embodiments, the integrated component may still perform one or more functions of each of the plurality of components in the same or similar manner as they are performed by a corresponding one of the plurality of components before the integration. According to various embodiments, operations performed by the module, the program, or another component may be carried out sequentially, in parallel, repeatedly, or heuristically, or one or more of the operations may be executed in a different order or omitted, or one or more other operations may be added.

According to one embodiment, the processor 120 may configure a health-care mode of the electronic device 101 on the basis of data related to health care. For example, when a user's preferred exercise and target weight are configured, the processor 120 may configure the health-care mode as a weight management mode to configure an exercise program corresponding to the preferred exercise. For example, when a user's preferred exercise is configured but a target weight is not configured, the processor 120 may configure the health-care mode as an exercise mode to configure an exercise program corresponding to the preferred exercise. For example, when a user's target weight is configured but the user's preferred exercise is not configured, the processor 120 may configure the health-care mode as a weight management mode. For example, when a user's preferred exercise and target weight are not configured, the processor 120 may configure the health-care mode as an exercise mode. In one example, the data related to the health care may include at least one among a user's profile, a use history of an electronic device, a user's preferred exercise information, a user's target weight, hospital visitation history (e.g. screening, medical records, etc.), and medicine purchase information. In one example, the weight management mode may include an operation mode for guiding a user to reach a target weight on the basis of the user's calorie-burning amount and calorie intake amount. In one example, the exercise mode may include an operation mode for measuring the amount of exercise by a user.

According to one embodiment, when the weight management mode is configured, the processor 120 may configure target calories for the management of a user's weight. For example, the processor 120 may configure a target calorie-burning amount and a target calorie intake amount on the basis of the user's current weight, the user's target weight, and a period of time for weight management. In one example, the processor 120 may adjust the ratio of the target calorie-burning amount and the target calorie intake amount on the basis of a weight management method. The weight management method may include a ratio of diet management and exercise for weight management. In one example, the target calorie-burning amount and the target calorie intake amount may include the amount of calories to burn or take in per day. For example, the target calories may include the amount of calories to additionally burn or additionally take in per day in order to reach the target weight within the target period of time. In one example, the target calories may be configured to be the difference between the target calorie-burning amount and the target calorie intake amount.

According to one embodiment, the processor 120 may identify a user's state related to the target weight. For example, the processor 120 may identify a duration and a type of motion on the basis of data acquired through the sensor module 176. The processor 120 may identify the user's calorie-burning amount on the basis of the duration and the type of motion. The processor 120 may identify information on the user's current state for reaching the target weight on the basis of the calorie intake amount and the calorie-burning amount of the user. In one example, the processor 120 may determine the difference between the calorie intake amount and the calorie-burning amount on the basis of the amount of the user's current calories. The processor 120 may control the display unit 160 to display information on a comparison between the user's current calorie amount and a target calorie amount at the current time point.

According to one embodiment, the processor 120 may provide guidance information for weight management on the basis of a user's state related to target weight. For example, when the amount of calories to additionally burn is detected by the comparison between the user's current calorie amount and the target calorie amount at the current time point, the processor 120 may determine exercise and the exercise time necessary to burn calories. The processor 120 may control the display unit 160 to display information on the exercise and the exercise time necessary to burn calories. In one example, the amount of calories to burn is the amount of calories which the user must additionally burn in order to meet the target calorie amount, and may be calculated as the difference between the target calorie amount and the current calorie amount.

According to one embodiment, the processor 120 may reconfigure a goal for weight management. For example, based on the comparison between the user's current calorie amount and the target calorie amount, the processor 120 may determine whether the goal is achieved. The processor 120 may reconfigure a weight management goal on the basis of whether the goal is achieved. In one example, when a goal achievement rate becomes lower than a reference value, the processor 120 may reconfigure at least one among the target weight, the target period of time, and the target calorie amount for weight management. In one example, when the number of times the goal is not achieved is larger a reference number of times, the processor 120 may reconfigure at least one among the target weight, the target period of time, the target calorie amount for weight management. In one example, the period in which whether the goal is achieved is determined may include a one-day unit.

According to various embodiments of the disclosure, the electronic device 101 may include a housing formed to surround the entirety or at least a part of the electronic device 101. For example, the display unit 160 may be disposed to be exposed through at least a part of the front plate of the housing. For example, at least some of the elements included in the electronic device 101 may be included in the housing.

According to various embodiments of the disclosure, the electronic device may include: a housing; a touch-screen display exposed through at least a part of the housing; a motion sensor disposed in the housing; a wireless communication circuit disposed in the housing; a processor disposed in the housing and electrically connected to the touch-screen display, the motion sensor, and the wireless communication circuit; and a memory disposed in the housing and electrically connected to the processor, wherein the memory may store instructions which cause, when executed, the processor to: acquire, using the motion sensor, data related to motion of a user carrying the electronic device; determine a duration and a type of the motion, based at least partially on the acquired data; acquire a first value related to the user's food intake; determine a second value indicating the amount of calories burned by the user, based at least partially on the duration and the type of the motion and a weighted value related to the type of the motion; determine a third value, based at least partially on the first value and the second value; and provide, based at least partially on the third value, a graphical user interface to the touch-screen display.

According to various embodiments, the third value may be the difference between the first value and the second value.

According to various embodiments, the graphical user interface may provide multiple ranges and may indicate that the third value belongs to one of the multiple ranges.

According to various embodiments, the electronic device may further include an instruction which causes the processor to determine the second value, based on the user's profile.

According to various embodiments, the electronic device may further include an instruction which causes the processor to determine the second value, based at least partially on the user's basal metabolic rate.

According to various embodiments, the electronic device may further include an instruction which causes the processor to determine the type of the motion, based at least partially on a user input received through the touch-screen display.

According to various embodiments, the electronic device may further include an instruction which causes the processor to provide the user interface to the touch-screen display so as to receive at least one user input corresponding to at least one of a type and amount or a calorie amount of food related to the food intake.

According to various embodiments, the electronic device may further include an instruction which causes the processor to: determine a weight management mode, based on at least one of the user's profile, a use history of the electronic device, or the user input; and configure target calories, based on the user's target weight.

According to various embodiments, the electronic device may further include an instruction which causes the processor to provide the graphical user interface to the touch-screen display, based on the target calories and the third value.

According to various embodiments, the electronic device may further include an instruction which causes the processor to: identify a target calorie intake amount corresponding to the user's target weight; and configure the first value, based on the target calorie intake amount.

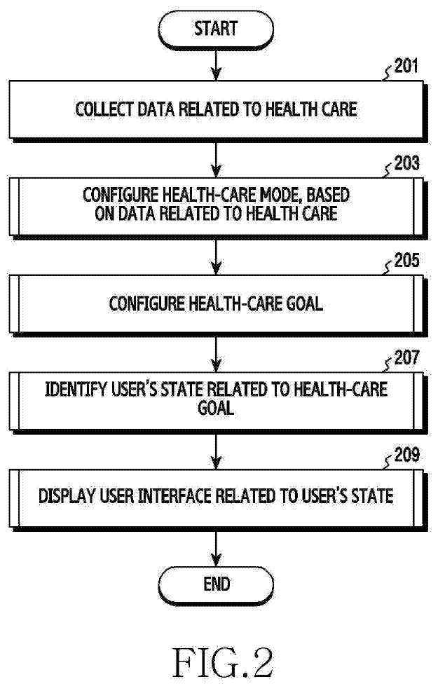

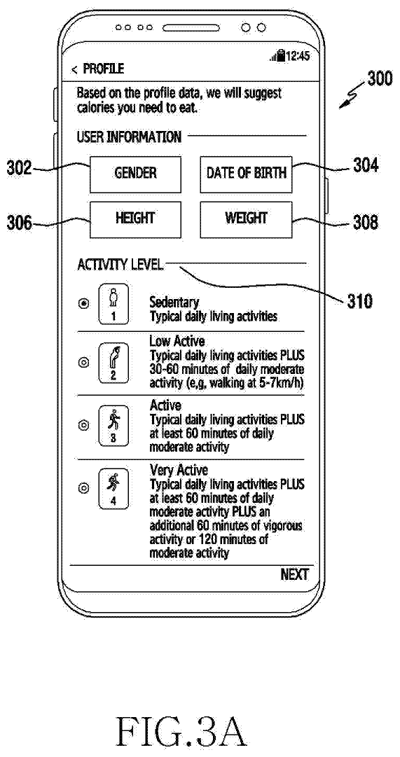

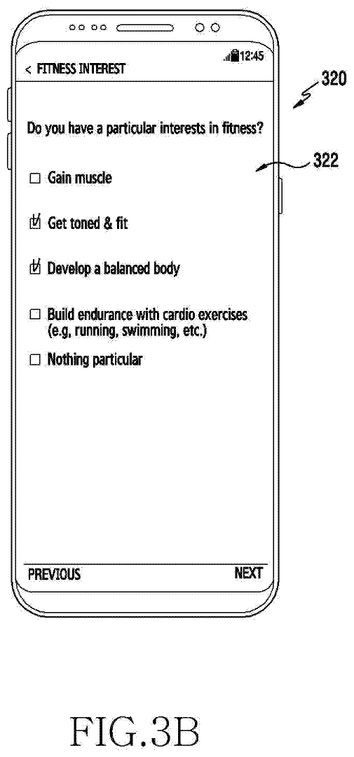

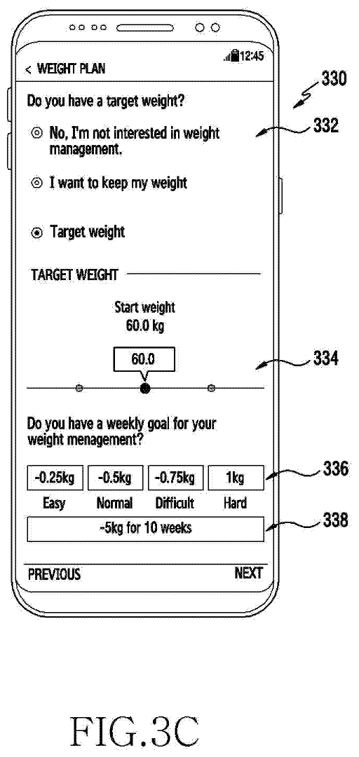

FIG. 2 illustrates a flowchart for displaying a user's state information at an electronic device according to various embodiments of the disclosure. FIGS. 3A to 3C illustrate a screen configuration of an electronic device for collecting data related to a user according to various embodiments of the disclosure. In the following description, the electronic device may include the electronic device 101 or at least a part (e.g. the processor 120) of the electronic device 101 in FIG. 1.

Referring to FIG. 2, in operation 201, the electronic device may collect data related to a user's health care. For example, when an event for data configuration occurs, the processor 120 may control the display unit 160 to display a user interface 300 for configuring a user profile, as shown in FIG. 3A. The processor 120 may identify a gender 302, a date of birth 304, a height 306, and a weight 308 on the basis of a user input received through the user interface 300 displayed on the display unit 160. In one example, an activity level 310 may be configured based on a user input or based on a history of a user's motion collected through the sensor module 176. For example, when input of a user profile is completed, the processor 120 may control the display unit 160 to display a user interface 320 for configuring preferred exercise, as shown in FIG. 3B. On the basis of a user input received through the user interface 320 for configuring preferred exercise, the processor 120 may select at least one exercise method or exercise type that a user prefers. For example, when the preferred exercise configuration is completed, the processor 120 may control the display unit 160 to display a user interface 330 for configuring a target weight, as shown in FIG. 3C. In one example, the user interface 330 for configuring the target weight may include a weight management plan 332, a target weight 334, a weight reduction level 336, and weight reduction information 338. In one example, the weight management plan 332 may include menu items for weight management non-configuration, weight maintenance configuration, and target weight configuration. The weight reduction level 336 may be automatically configured based on the target weight 334 and a target period of time, or may be configured based on a user input. In one example, data related to a user's health care may further include at least one among a user's hospital visitation history (e.g. screening, medical records, etc.), medicine purchase information, or schedule information.

In operation 203, the electronic device may configure a health-care mode on the basis of the data related to the health care. For example, the processor 120 may select a health-care mode corresponding to one of a weight management mode or an exercise mode on the basis of at least one of a preferred exercise or a target weight.

In operation 205, the electronic device may configure a health-care goal on the basis of the health-care mode. For example, when the weight management mode is selected as the health-care mode of the electronic device 101, the processor 120 may configure a daily target calorie amount for reaching a target weight in a target period of time. In one example, the target calorie amount may include a target calorie-burning amount and a target calorie intake amount.

In operation 207, the electronic device may identify the user's state related to the health-care goal. For example, the processor 120 may detect the user's current calorie amount on the basis of the user's calorie-burning amount and calorie intake amount. The processor 120 may identify the user's state related to the health-care goal on the basis of a target calorie amount at the current time point and the user's current calorie amount. In one example, the calorie-burning amount may be detected based on sensor data collected through the sensor module 176 and the user's basal metabolic rate at the current time point. In one example, the calorie intake amount may be configured based on a user input or may be automatically configured based on the target calorie intake amount. In one example, the target calorie amount at the current time point may include a calorie amount obtained by updating a daily configured target calorie amount on the basis of the current time point. In one example, the basal metabolic rate at the current time point may include a value obtained by updating a daily configured basal metabolic rate of the user on the basis of the current time point.

In operation 209, the electronic device may display a user interface related to the user's state corresponding to the health-care goal. For example, the user's state corresponding to the health-care goal may include whether the target calorie amount is achieved based on the user's current calorie amount. In one example, the user interface related to the user's state corresponding to the health-care goal may include information on achievement of the target calorie amount on a daily, weekly, or monthly basis.

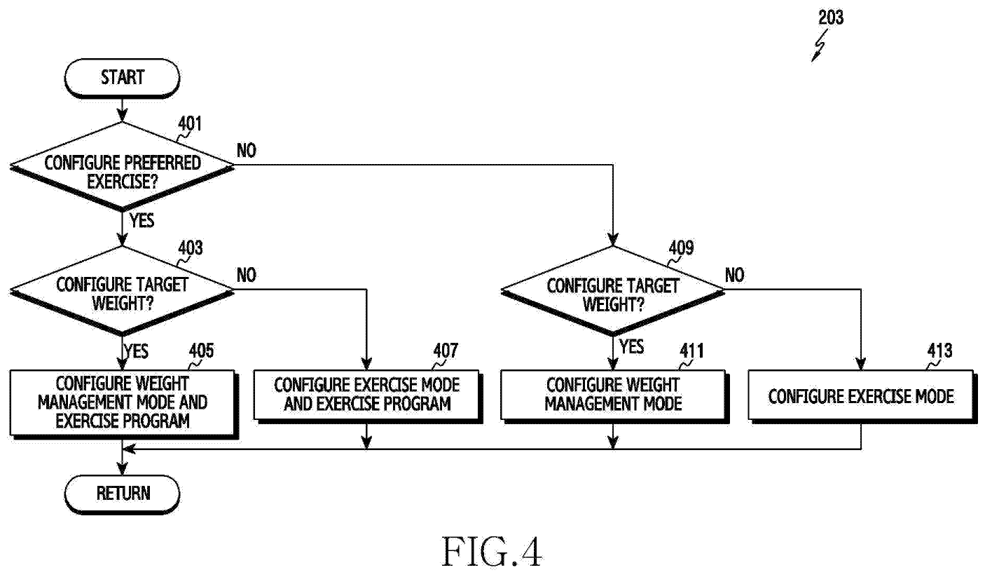

FIG. 4 illustrates a flowchart for configuring a weight management mode in an electronic device according to various embodiments of the disclosure. Hereinafter, a description will be made of the configuration of a health-care mode in operation 203 of FIG. 2. In the following description, the electronic device may include the electronic device 101 or at least a part (e.g. the processor 120) of the electronic device 101 in FIG. 1.

Referring to FIG. 4, in operation 401, when data related to a user's health care is collected (e.g. operation 201 in FIG. 2), the electronic device may identify whether the user's preferred exercise has been configured. For example, the processor 120 may identify whether the user's preferred exercise has been configured through the user interface 320 for a preferred exercise configuration in FIG. 3B.

In operation 403, when the user's preferred exercise is configured, the electronic device may identify whether the user's target weight has been configured. For example, when at least one item other than “there is no preferred exercise (nothing particular)” is selected in the user interface 320 for the preferred exercise configuration in FIG. 3B, the processor 120 may determine that the user's preferred exercise has been configured. The processor 120 may identify whether the user's target weight has been configured through the user interface 330 for a target weight configuration in FIG. 3C.

In operation 405, when the user's target weight is configured, the electronic device may configure the health-care mode of the electronic device as a weight management mode, and may configure an exercise program corresponding to the preferred exercise. For example, when a weight maintenance menu or a target weight configuration menu is selected in the weight management plan 332 of the user interface 320 for the target weight configuration in FIG. 3C, the processor 120 may determine that the user's target weight has been configured. When the user's preferred exercise or target weight is configured, the processor 120 may configure an exercise program corresponding to the weight management mode and the preferred exercise. In one example, when the weight maintenance menu is selected, the user's target weight may be configured based on the user's current weight input through the user interface 300 for configuring the user profile in FIG. 3A. In one example, the exercise program corresponding to the preferred exercise may include at least one of guidance information regarding a method for performing the preferred exercise or schedule information to be executed so as to reach the target weight by using the preferred exercise.

In operation 407, when the user's target weight is not configured, the electronic device may configure the health-care mode of the electronic device as an exercise mode, and may configure an exercise program corresponding to the preferred exercise. For example, when a weight management non-configuration menu (“No, I'm not interested in weight management”) is selected in the weight management plan 332 of the user interface 330 for the target weight configuration in FIG. 3C, the processor 120 may determine that the user's target weight has not been configured. When the user's preferred exercise is configured but the target weight is not configured, the processor 120 may configure an exercise program corresponding to the exercise mode and the preferred exercise.

In operation 409, when the user's preferred exercise is not configured, the electronic device may identify whether the user's target weight has been configured. For example, when an item “there is no preferred exercise (nothing particular)” is selected in the user interface 320 for the preferred exercise configuration in FIG. 3B, the processor 120 may determine that the user's preferred exercise has not been configured.

In operation 411, when the user's target weight is configured, the electronic device may configure the health-care mode of the electronic device as the weight management mode. For example, when the user's preferred exercise is not configured and the target weight is configured, the processor 120 may configure the health-care mode of the electronic device 101 as the weight management mode.

In operation 413, when the user's target weight is not configured, the electronic device may configure the health care management mode of the electronic device as the exercise mode. For example, when the user's preferred exercise and target weight are not configured, the processor 120 may configure the health-care mode of the electronic device 101 as the exercise mode.

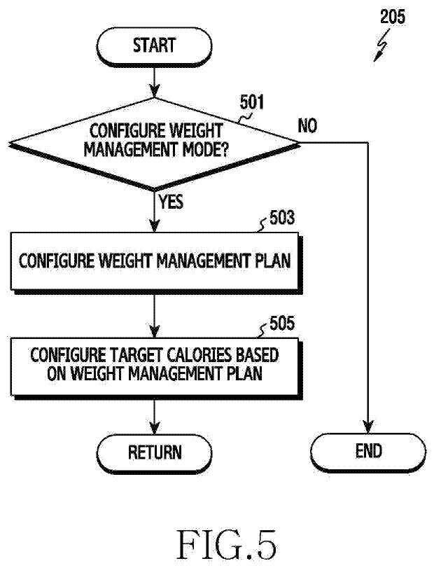

FIG. 5 illustrates a flowchart for configuring target calories in an electronic device according to various embodiments of the disclosure. Hereafter, a description will be made of the configuration of the health-care goal in operation 205 of FIG. 2. In the following description, the electronic device may include the electronic device 101 or at least a part (e.g. the processor 120) of the electronic device 101 in FIG. 1.

Referring to FIG. 5, in operation 501, when a health-care mode of the electronic device has been configured (e.g. operation 203 in FIG. 2), the electronic device may identify whether the health-care mode has been configured to be a weight management mode. For example, when a user's target weight is configured, the processor 120 may determine that the health-care mode of the electronic device 101 has been configured to be a weight management mode.

When the health-care mode is configured to be an exercise mode, the electronic device may determine that a target calorie amount for weight management is not configured.

In operation 503, when the health-care mode of the electronic device is configured to be the weight management mode, the electronic device may configure a plan for the user' weight management on the basis of the user's current weight or target weight. For example, when the current weight equals the target weight, the processor 120 may select weight maintenance as a plan for weight management. When the current weight is greater than the target weight, the processor 120 may select a weight reduction as a plan for weight management. When the current weight is smaller than the target weight, the processor 120 may select a weight increase as a plan for weight management.

In operation 505, the electronic device may configure the target calories on the basis of a weight management plan. For example, the processor 120 may configure the user's daily target calorie-burning amount and target calorie intake amount such that the user's weight reaches the target weight. The target calories may be configured by the difference between the target calorie-burning amount and the target calorie intake amount. In one example, the processor 120 may configure the target calories as in Table 1 below.

TABLE 1

| Health- | Weight | |
|---|---|---|
| care | management | |
| mode | plan | Target calories |
| Weight | Weight | Target calorie-burning amount = EER + |
| management | reduction | (DTC/2) |
| mode | | Target calorie intake amount = EER − |
| | | (DTC/2) |
| | | Maximum calorie-burning amount = |
| | | EER + DTC, Minimum calorie-burning |
| | | amount = EER |
| | | Maximum calorie intake amount = EER, |
| | | Minimum calorie intake amount = |
| | | EER − DTC |
| | Weight | Target calorie-burning amount = EER |
| | maintenance | Target calorie intake amount = EER |
| | Weight | Target calorie-burning amount = EER − |
| | increase | (DTC/2) |
| | | Target calorie intake amount = EER + |
| | | (DTC/2) |
| | | Maximum calorie-burning amount = EER, |
| | | Minimum calorie-burning amount = |
| | | EER − DTC |
| | | Maximum calorie intake amount = |
| | | EER + DTC, Minimum calorie intake |
| | | amount = EER |

In Table 1, the estimated energy requirement (EER) may indicate a calorie intake amount necessary for maintaining an energy balance of a body activity level corresponding to a user's age, gender, height, weight, and health state. The daily target calories (DTC) may indicate a weight change amount corresponding to a calorie amount on a daily basis.

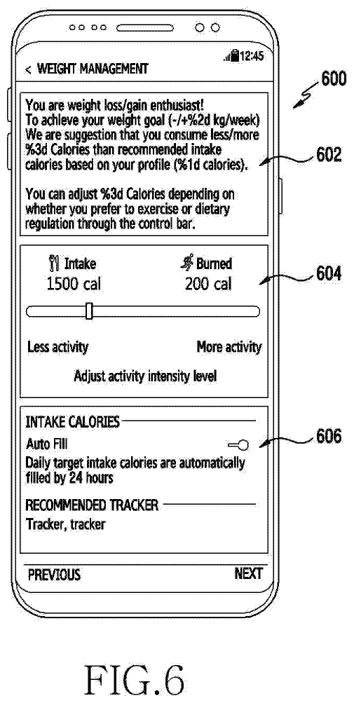

FIG. 6 illustrates a screen configuration of an electronic device for configuring target calories according to various embodiments of the disclosure. In the following description, the electronic device may include the electronic device 101 or at least a part (e.g. the processor 120) of the electronic device 101 in FIG. 1.

According to one embodiment, the electronic device may adjust the ratio of a target calorie-burning amount and a target calorie intake amount on the basis of a weight management method configured based on a user input. For example, when target calories for a user's weight management are configured, the processor 120 may control the display unit 160 to display a user interface 600 corresponding to the weight management method, as shown in FIG. 6. The user interface 600 may include a weight management guidance wording 602, a weight management method configuration menu 604, and a calorie intake amount auto-configuration menu 606.

According to one embodiment, the electronic device may determine relative importance of exercise and diet management for weight management on the basis of a user input corresponding to the weight management method configuration menu 604 illustrated in FIG. 6. For example, when the processor 120 determines the relative importance of exercise to be higher than that of diet management for weight management, the target calorie-burning amount and the target calorie intake amount may be relatively increased. For example, when the processor 120 determines the relative importance of diet management to be higher than that of exercise for weight management, the target calorie-burning amount and the target calorie intake amount may be relatively decreased.

According to one embodiment, the electronic device may determine whether to automatically configure a calorie intake amount on the basis of a user input corresponding to the calorie intake amount auto-configuration menu 606 illustrated in FIG. 6. For example, when the calorie intake amount auto-configuration menu 606 is activated, the processor 120 may automatically configure, based on the target calorie intake amount, a user's calorie intake amount for at least one among the morning, midday, or evening.

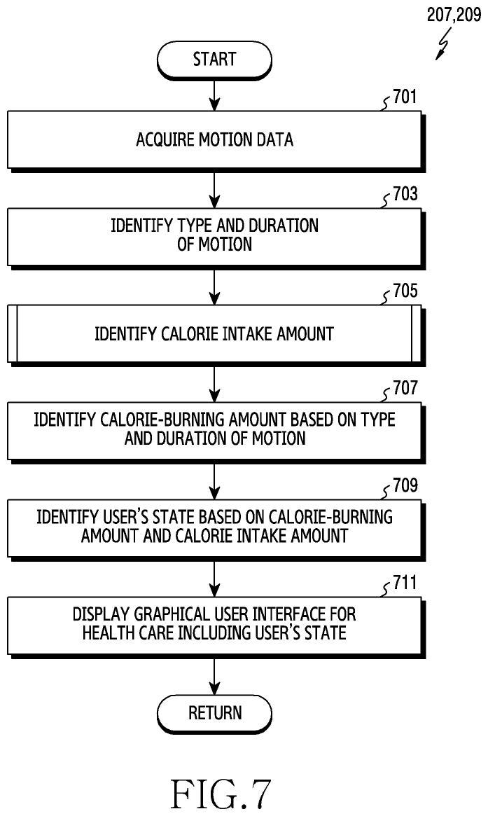

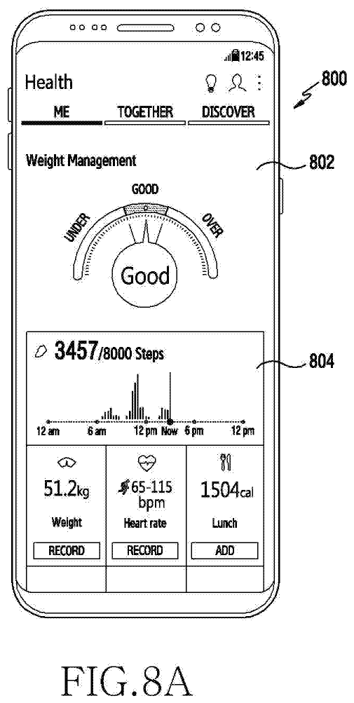

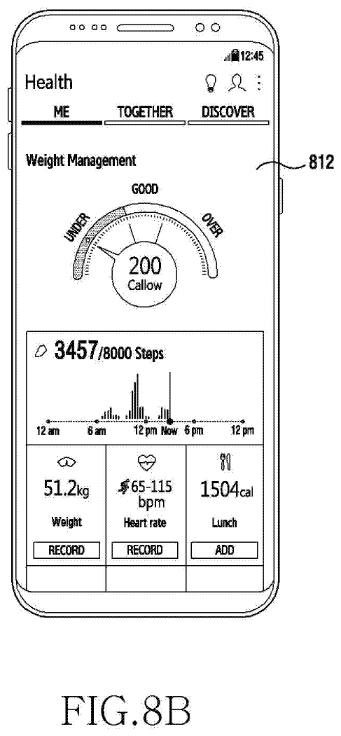

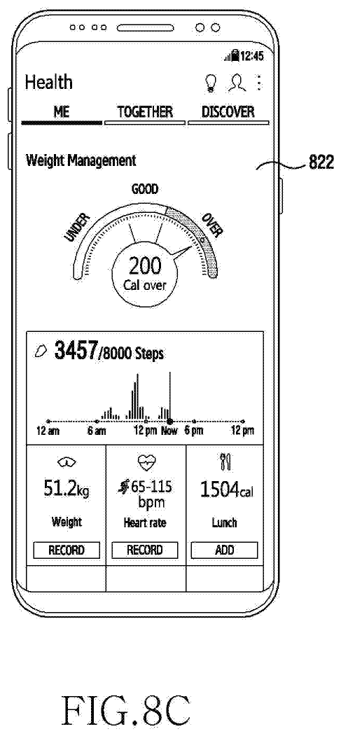

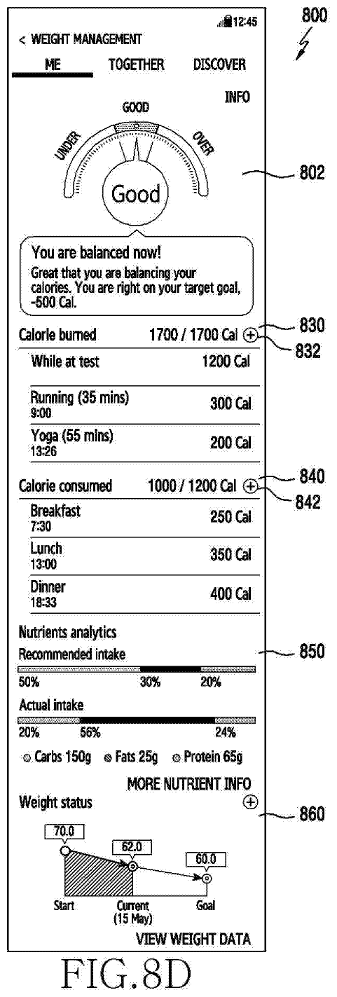

FIG. 7 illustrates a flowchart for displaying a user's state information for weight management in an electronic device according to various embodiments of the disclosure. FIGS. 8A to 8D illustrate a configuration of a user interface including a user's state information on a daily basis in an electronic device according to various embodiments of the disclosure. Hereinafter, a description will be made of the displaying of a user's state related to a health-care goal in operation 207 and operation 209 of FIG. 2. In the following description, the electronic device may include the electronic device 101 or at least a part (e.g. the processor 120) of the electronic device 101 in FIG. 1.

In operation 701, when a user's health-care goal is configured (e.g. operation 205 in FIG. 2), the electronic device may acquire motion data of the electronic device. For example, the processor 120 may acquire sensor data corresponding to the motion of the electronic device 101 through the sensor module 176. In one example, the sensor data may include data related to the motion of the user carrying the electronic device 101.

In operation 703, the electronic device may identify the duration and type of the motion on the basis of the motion data of the electronic device. For example, the processor 120 may extract patterns repeatedly detected from the sensor data acquired from the sensor module 176. The processor 120 may determine a motion type on the basis of at least one among the waveform, size, interval, or period of the repeatedly detected patterns. In one example, the motion type may include the user's motion type, such as walking, running, cycling, etc.

In operation 705, the electronic device may acquire a first value related to the user's food intake. For example, the processor 120 may identify a calorie intake amount related to the user's food intake on the basis of a user input received through the input unit 150. For example, when the calorie intake amount auto-configuration menu 606 is activated as shown in FIG. 6, the processor 120 may automatically configure the calorie intake amount of at least one among the morning, midday, or evening. In one example, the automatically configured calorie intake amount may be configured based on a target calorie intake amount.

In operation 707, the electronic device may identify a second value indicating the user's calorie-burning amount on the basis of the duration and type of the motion. For example, the processor 120 may calculate the user's calorie-burning amount on the basis of at least one among the user's basal metabolic rate at the current time point, an exercise amount corresponding to the duration and type of the motion, or a calorie-burning amount attributable to food intake. In one example, the basal metabolic rate at the current time point may include a value obtained by updating the user's basal metabolic rate, which has been configured daily based on the user's weight, height, age, and gender, so as to correspond to the current time point. In one example, the exercise amount may be determined based on the duration of the motion and a weighted value corresponding to the type of the motion. In one example, the calorie-burning amount attributable to food intake may include a calorie amount included in a reference percentage (e.g. 10%) of the calorie intake amount.

In operation 709, the electronic device may determine the user's state corresponding to the user's weight management on the basis of the calorie-burning amount and the calorie intake amount. In one example, a third value corresponding to the user's weight management may include the difference between the user's calorie-burning amount and calorie intake amount. For example, the processor 120 may identify the user's state related to a target weight on the basis of a target calorie amount and the third value corresponding to the user's weight management. In one example, when the difference between the target calorie amount and the third value corresponding to the user's weight management is included in a reference range (e.g. ±25 kcal), the processor 120 may determine that the weight management goal is achieved at the current time point. In one example, when the third value corresponding to the user's weight management is greater than the target calorie amount by the reference range (e.g. ±25 kcal) or more, the processor 120 may determine that additional burning of calories is required. In one example, when the target calorie amount is greater than the third value corresponding to the user's weight management at or beyond the reference range (e.g. ±25 kcal) or more, the processor 120 may determine that the calorie-burning amount exceeds the goal.

In operation 711, the electronic device may display a graphical user interface (GUI) for health care including the user's state corresponding to the weight management. For example, when it is determined that the weight management goal is achieved at the current time point, the processor 120 may control the display unit 160 to display calorie state information as “good” 802 in a user interface 800 for health care, as shown in FIG. 8A. In one example, the user interface 800 for health care may include an exercise amount 804 corresponding to the user's motion type. For example, when it is determined that the calorie-burning amount exceeds the goal, the processor 120 may control the display unit 160 to display calorie state information as “under” 812 in the user interface 800 for health care, as shown in FIG. 8B. In one example, when it is determined that additional burning of calories is required, the processor 120 may control the display unit 160 to display calorie state information as “over” 822 in the user interface 800 for health care as in FIG. 8C. “Good” 802, “under” 812, and “over” 822, indicating calorie state information, may be included in the graphical user interface.

According to one embodiment, as in FIG. 8D, the user interface 800 for health care may further include a calorie-burning amount 830, a calorie intake amount 840, nutrition analysis information 850, and weight change information 860. For example, the calorie-burning amount 830 may include a target calorie-burning amount, a current calorie-burning amount, and a calorie-burning amount according to each motion type. In one example, the processor 120 may input a calorie-burning amount through the user's direct input on the basis of input of an activity log addition menu 832, or may connect an exercise menu for calorie burning. For example, the calorie intake amount 840 may include a target calorie intake amount, a current calorie intake amount, and a calorie intake amount every time food is eaten. In one example, the calorie intake amount every time food is eaten may include an input type (e.g. an automatic input) of a calorie intake amount. For example, the weight change information 860 may include the user's weight change graph down or up to the target weight. In one example, when the target weight is reconfigured, the weight change information 860 may be changed based on the reconfigured target weight.

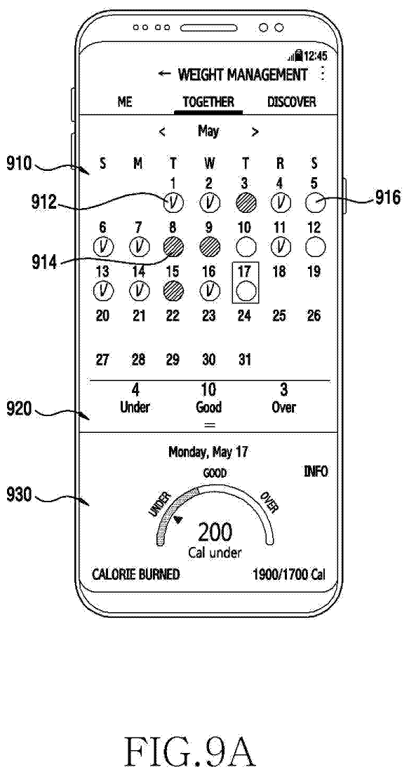

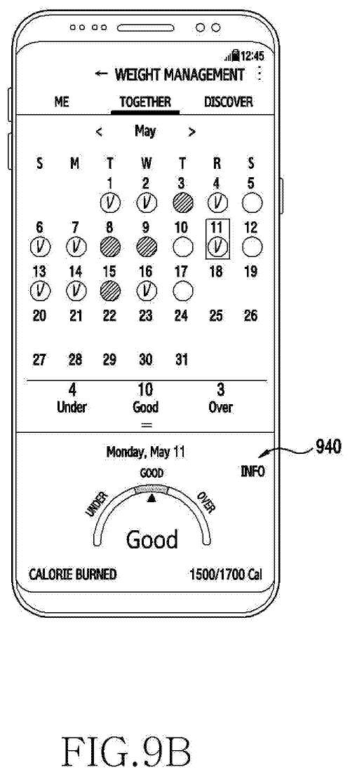

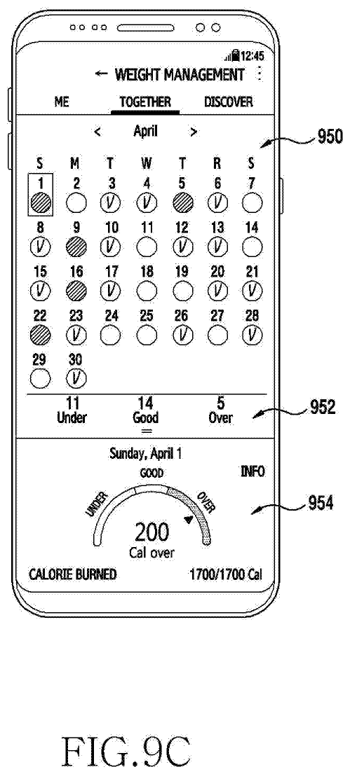

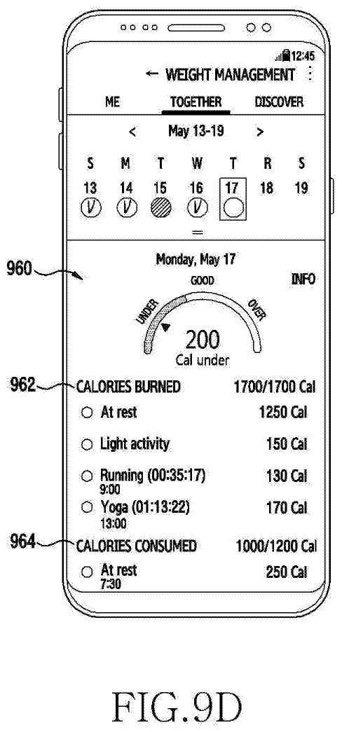

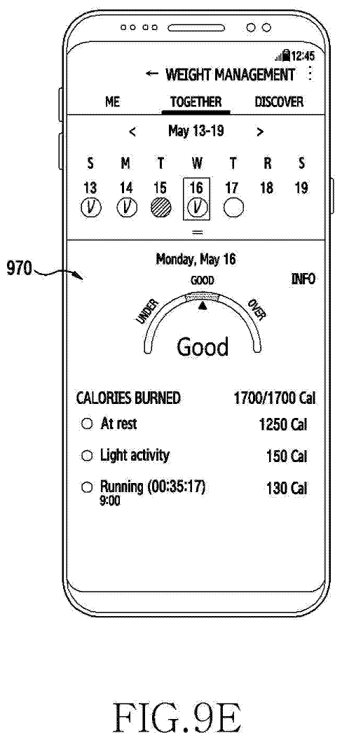

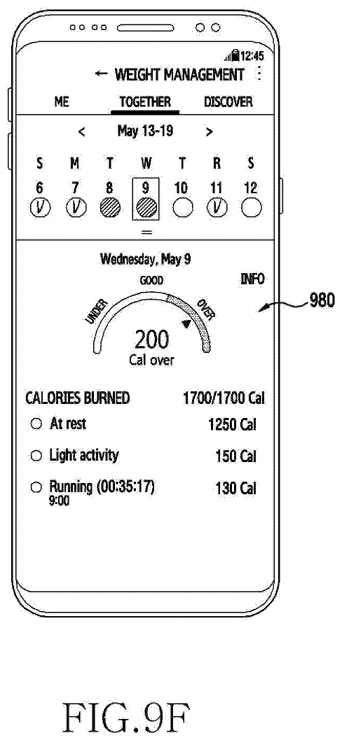

FIGS. 9A to 9F illustrate a configuration of a user interface including a user's state information on a monthly basis in an electronic device according to various embodiments of the disclosure. In the following description, the electronic device may include the electronic device 101 or at least a part (e.g. the processor 120) of the electronic device 101 in FIG. 1.

According to one embodiment, the electronic device may display a user interface 900 for health care on a monthly basis, as shown in FIG. 9A. For example, the user interface 900 for health care may include daily goal achievement information 910, goal summary information 920, and a calorie gauge 930. In one example, the daily goal achievement information 910 may indicate whether a daily target is achieved, using an achievement 912, a calorie surplus 914, and a calorie deficit 916. In one example, the goal summary information 920 may include the number of days in which the achievement 912, the calorie surplus 914, and the calorie deficit 916 occur in the corresponding month. In one example, the calorie gauge 930 may display, in a gauge form, goal achievement information for a date selected on a calendar.

According to one embodiment, as in FIG. 9B, the electronic device may change a date corresponding to the calorie gauge on the basis of a user input (e.g. a tap input). For example, when a specific date (e.g. the 11th) is selected on a calendar displayed on the display unit 160, the processor 120 may control the display unit 160 such that goal achievement information of the corresponding date is displayed through a calorie gauge 940.

According to one embodiment, as shown in FIG. 9C, the electronic device may change, based on a user input (e.g. swipe input), the month for which the monthly goal achievement information is displayed through the user interface 900 for health care. For example, when a swipe input is received in the state in which the goal achievement information for May is displayed, as shown in FIG. 9A, the processor 120 may control the display unit 160 to display goal achievement information for April, as shown in FIG. 9C. In one example, the display unit 160 may display daily goal achievement information 950 for April, goal summary information 952 for April, and a calorie gauge 954 for April 1. In one example, the processor 120 may determine, based on a swipe direction, the month for which goal achievement information is to be displayed on the display unit 160.

According to one embodiment, as shown in FIG. 9D, the electronic device may display, based on a user input, detailed information corresponding to goal achievement on a specific date. For example, when an event for viewing details occurs, the processor 120 may control the display unit 160 to display a calorie gauge 960, a calorie-burning amount 962, and a calorie intake amount 964 for a specific date (e.g. May 17), as shown in FIG. 9D. In one example, the event for viewing details may occur based on at least one among a swipe input in the upward direction, an input on a hardware button, or a selective input of an icon corresponding to viewing details.

According to one embodiment, as in FIG. 9E, the electronic device may change, based on a user input (e.g. a tap input), the date for which detailed information corresponding to goal achievement is displayed. For example, when a specific date (e.g. the 16th) is selected on a calendar displayed on the display unit 160, the processor 120 may control the display unit 160 to display detailed information 970 corresponding to goal achievement for the corresponding date.

According to one embodiment, as shown in FIG. 9F, the electronic device may change, based on a user input (e.g. a swipe input), the week for which detailed information corresponding to goal achievement is displayed. For example, when a swipe input is received in the state in which detailed information corresponding to goal achievement in the third week (the 13th to 19th) of May is displayed as shown in FIG. 9E, the processor 120 may control the display unit 160 to display detailed information 980 corresponding to goal achievement for the second week of May, as shown in FIG. 9F.

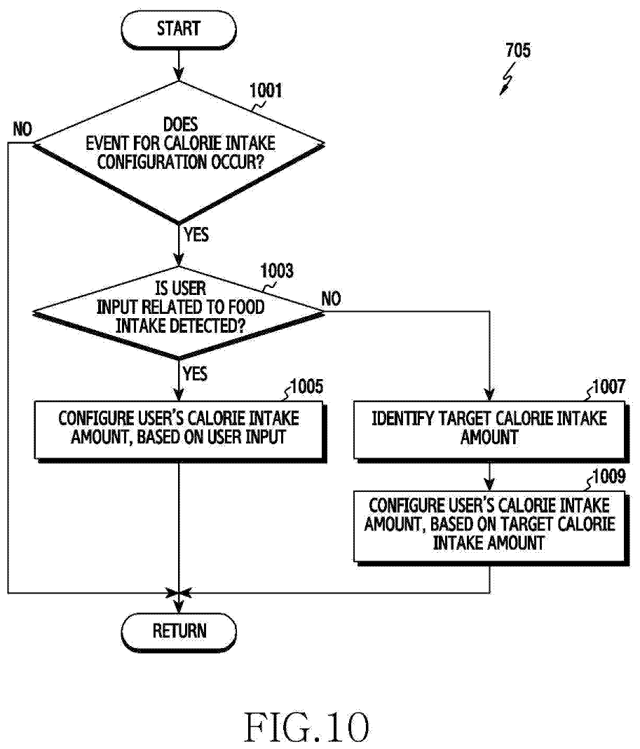

FIG. 10 illustrates a flowchart for identifying a calorie intake amount in an electronic device according to various embodiments of the disclosure. Hereinafter, a description will be made of the identification of the calorie intake amount in operation 705 of FIG. 7. In the following description, the electronic device may include the electronic device 101 or at least a part (e.g. the processor 120) of the electronic device 101 in FIG. 1.

Referring to FIG. 10, in operation 1001, the electronic device may identify whether an event for configuring a calorie intake amount occurs. For example, the processor 120 may identify whether a calorie intake time point (e.g. the morning, midday, or evening) has arrived.

In operation 1003, when the event for configuring a calorie intake amount has occurred, the electronic device may identify whether a user input related to food intake is detected. For example, the processor 120 may receive information on food taken in by a user through the input unit 150, or may identify whether food intake information is received from an external device.

In operation 1005, when the user input related to the food intake has been detected, the electronic device may configure the user's calorie intake amount on the basis of the user input. For example, the processor 120 may configure a calorie amount corresponding to the type and amount of food selected by the user as the user's calorie intake amount.

In operation 1007, when the user input related to the food intake has not been detected, the electronic device may identify a target calorie intake amount for the user's weight management. For example, the processor 120 may identify the daily target calorie intake amount configured in operation 205 of FIG. 2.

In operation 1009, the electronic device may configure, based on the target calorie intake amount, the user's calorie intake amount at a pertinent time point. For example, the processor 120 may divide a target calorie intake amount into the number of time points for configuring a calorie intake amount, and may configure the user's calorie intake amount corresponding to each corresponding time point. For example, the processor 120 may configure the user's calorie intake amount corresponding to each corresponding time point on the basis of the number of calorie intake amount configuration time points into which the target calorie intake amount is divided and a weighted value at each calorie intake amount configuration time point. In one example, the weighted value is configured such that a calorie intake amount in the morning is configured to be relatively lower than those at midday and in the evening, and the weighted value may be configured such that a calorie intake amount at midday is configured to be relatively higher than those in the morning and in the evening. The weighted value may be updated based on a configuration history at a configuration time point of a calorie intake amount.

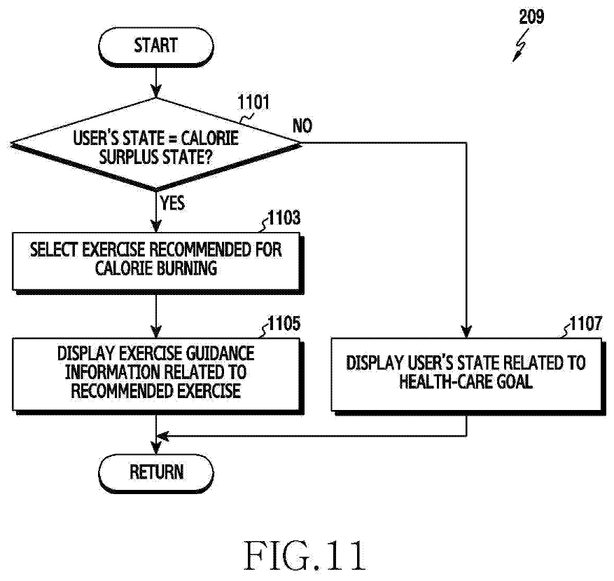

FIG. 11 illustrates a flowchart for displaying exercise guidance information in an electronic device according to various embodiments of the disclosure. Hereinafter, a description will be made of the display of the user interface related to the user's state corresponding to health care in operation 209 of FIG. 2. In the following description, the electronic device may include the electronic device 101 or at least a part (e.g. the processor 120) of the electronic device 101 in FIG. 1.

Referring to FIG. 11, in operation 1101, when a user's state related to a health-care goal is identified (e.g. operation 207 in FIG. 2), the electronic device may identify whether the user's state related to the health-care goal is a calorie surplus state. For example, the processor 120 may calculate the difference between the user's calorie-burning amount and calorie intake amount. When the difference between the calorie-burning amount and the calorie intake amount is more than a target calorie amount, the processor 120 may determine that the user's state related to the health-care goal is the calorie surplus state.

In operation 1103, when the user's state related to the health-care goal is the calorie surplus state, the electronic device may select a recommended exercise for calorie burning. For example, when the user's state related to the health-care goal is the calorie surplus state, the processor 120 may determine an exercise to recommend for the user's calorie burning on the basis of the remaining activity time and surplus calorie amount of the user. In one example, as the user's activity time is shorter or the number of surplus calories is higher, a relatively higher-intensity exercise may be selected as the recommended exercise. In one example, the remaining time of the user's activity may include the time remaining until bedtime determined by the sleep pattern of the user.

In operation 1105, the electronic device may display exercise guidance information related to the recommended exercise for calorie burning. For example, the exercise guidance information related to the recommended exercise may include at least one among a type of the recommended exercise, a method for performing the recommended exercise, or a recommended exercise time.

In operation 1107, when the user's state related to the health-care goal corresponds to goal achievement or a calorie deficit, the electronic device may display the user's state related to the health-care goal. For example, when the user's state related to the health-care goal corresponds to goal achievement, the processor 120 may control the display unit 160 to display the user interface 800 including the calorie gauge 802 corresponding to the goal achievement, as shown in FIG. 8A. For example, when the user's state related to the health-care goal corresponds to the calorie deficit, the processor 120 may control the display unit 160 to display the user interface 800 including the calorie gauge 812 corresponding to the calorie deficit, as shown in FIG. 8B.

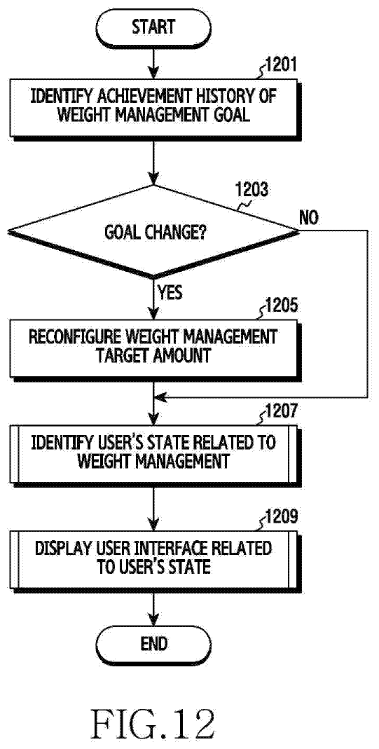

FIG. 12 illustrates a flowchart for reconfiguring a target weight amount in an electronic device according to various embodiments of the disclosure. In the following description, the electronic device may include the electronic device 101 or at least a part (e.g. processor 120) of the electronic device 101 in FIG. 1.

Referring to FIG. 12, in operation 1201, the electronic device may identify an achievement history of a weight management goal. For example, the processor 120 may identify daily goal achievement information from the time point at which a weight management mode stored in the memory 130 is executed.

In operation 1203, the electronic device may identify whether an event for goal reconfiguration occurs, based on the weight management goal achievement history. For example, when a goal achievement rate is equal to or smaller than a reference value, the processor 120 may determine that the event for goal reconfiguration has occurred. For example, when the number of times the goal is not achieved is equal to or smaller than a reference number, the processor 120 may determine that the event for goal reconfiguration has occurred.

When the event for goal reconfiguration has not occurred, the electronic device may identify the user's state related to the previously configured target weight in operation 1207.

When the event for the goal reconfiguration has occurred, the electronic device may reconfigure a target amount for weight management in operation 1205. For example, when the event for the goal reconfiguration has occurred, the processor 120 may change the user's target weight and a target period of time to a predefined unit. For example, when the event for goal reconfiguration has occurred, the processor 120 may control the display unit 160 to display a user interface for the goal reconfiguration. The processor 120 may reconfigure the user's target weight and the target period of time on the basis of a user input received through the user interface for the goal reconfiguration.

In operation 1207, the electronic device may identify the user's state related to the weight management. For example, the processor 120 may detect the user's current calorie amount on the basis of the user's calorie-burning amount and calorie intake amount. The processor 120 may compare a target calorie amount at the current time point with the user's current calorie amount to identify the user's state for weight management. In one example, the user's state for weight management may include one among goal achievement, calorie surplus, or calorie deficit.

In operation 1209, the electronic device may display a user interface related to the user's state corresponding to weight management. For example, as in operations 701 to 711 of FIG. 7, the processor 120 may control the display unit 160 to display a user interface including user state information related to the weight management. In one example, the user interface may be displayed on the display unit 160 in the form illustrated in one of FIGS. 8A to 8D or FIGS. 9A to 9F.

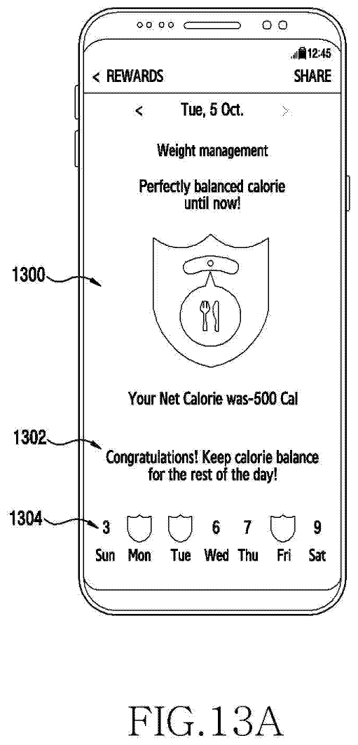

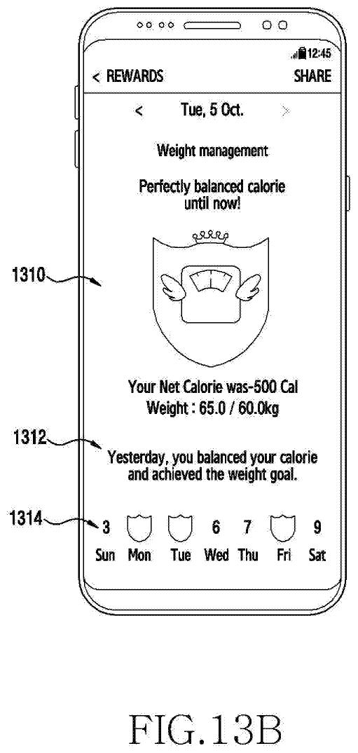

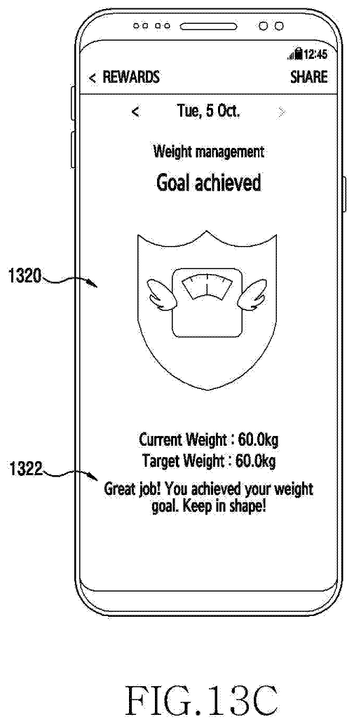

FIGS. 13A to 13C illustrate a screen configuration of an electronic device corresponding to achieving a weight management goal according to various embodiments of the disclosure. In the following description, the electronic device may include the electronic device 101 or at least a part (e.g. the processor 120) of the electronic device 101 in FIG. 1.

According to one embodiment, when the weight management goal has been achieved, the electronic device may provide a reward for achievement of the weight management goal, as shown in FIGS. 13 to 13C. For example, when the weight management goal has been achieved, the processor 120 may control the display unit 160 to display a screen including a first icon 1300 corresponding to goal achievement, target calorie information 1302, and weekly goal achievement information 1304 as in FIG. 13A. For example, when the weight management goal has been achieved, the processor 120 may control the display unit 160 to display a screen including a second icon 1310 corresponding to goal achievement, target calorie and weight information 1312, and weekly goal achievement information 1314, as shown in FIG. 13B. For example, when the weight management goal has been achieved, the processor 120 may control the display unit 160 to display a screen including a third icon 1320 corresponding to goal achievement and target weight information 1322, as shown in FIG. 13C.

According to various embodiments of the disclosure, an operation method of an electronic device may include: acquiring data related to motion of a user carrying the electronic device; determining a duration and a type of the motion, based at least partially on the acquired data; acquiring a first value related to the user's food intake; determining a second value indicating the amount of calories burned by the user, based at least partially on the duration and the type of the motion and a weighted value related to the type of the motion; determining a third value, based at least partially on the first value and the second value; and providing, based at least partially on the third value, a graphical user interface to a touch-screen display electrically connected to the electronic device.

According to various embodiments, the third value may be the difference between the first value and the second value.

According to various embodiments, the graphical user interface may provide multiple ranges and may indicate that the third value belongs to one of the multiple ranges.

According to various embodiments, the determining of the second value may include determining the second value, based on the user's profile.

According to various embodiments, the determining of the second value may include determining the second value, based at least partially on the user's basal metabolic rate.

According to various embodiments, the determining of the type of the motion may include determining the type of the motion, based at least partially on a user input received through the touch-screen display.

According to various embodiments, the operation method may further include providing the user interface to the touch-screen display so as to receive at least one user input corresponding to at least one of a type and amount or a calorie amount of food related to the food intake.

According to various embodiments, the operation method may further include: determining a weight management mode, based on at least one of the user's profile, a use history of the electronic device, or the user's input; and configuring target calories, based on the user's target weight.

According to various embodiments, the operation method may further include providing the graphical user interface to the touch-screen display, based on the target calories and the third value.

According to various embodiments, the acquiring of the first value may include: identifying a target calorie intake amount corresponding to the user's target weight; and configuring the first value based on the target calorie intake amount.

Meanwhile, various embodiments of the disclosure have been described, but various changes in form and details may be made therein without departing from the scope of the various embodiments of the disclosure. Therefore, the scope of the disclosure should not be defined as being limited to the embodiments described above, but should be defined by the appended claims and equivalents thereof.

1. An electronic device comprising:
a housing;
a touch-screen display exposed through at least a part of the housing;
a motion sensor disposed in the housing;
a wireless communication circuit disposed in the housing;
a processor disposed in the housing and electrically connected to the touch-screen display, the motion sensor, and the wireless communication circuit; and
a memory disposed in the housing and electrically connected to the processor,
wherein the memory stores instructions which cause, when executed, the processor to:
acquire, using the motion sensor, data related to motion of a user carrying the electronic device;
determine a duration and a type of the motion, based at least partially on the acquired data;
acquire a first value related to a food intake of the user;
determine a second value indicating an amount of calories burned by the user, based at least partially on the duration and the type of the motion and a weighted value related to the type of the motion; and
provide, based at least partially on the first value and the second value, a graphical user interface to the touch-screen display.

2. The electronic device of claim 1, further comprising an instruction which causes the processor to provide the graphical user interface to the touch-screen display, based at least partially on a third value corresponding to a difference between the first value and the second value.

3. The electronic device of claim 2, wherein the graphical user interface provides multiple ranges and indicates that the third value belongs to one of the multiple ranges.

4. The electronic device of claim 1, further comprising an instruction which causes the processor to determine the second value, based at least partially on a profile of the user or a basal metabolic rate of the user.

5. The electronic device of claim 1, further comprising an instruction which causes the processor to determine the type of the motion, based at least partially on a user input received through the touch-screen display.

6. The electronic device of claim 1, further comprising an instruction which causes the processor to provide the user interface to the touch-screen display so as to receive at least one user input corresponding to at least one of a type and amount or a calorie amount of food related to the food intake.

7. The electronic device of claim 1, further comprising an instruction which causes the processor to:
determine a weight management mode, based on at least one of the profile of the user, a use history of the electronic device, or the user input; and
configure target calories, based on a target weight of the user.

8. The electronic device of claim 7, further comprising an instruction which causes the processor to provide the graphical user interface to the touch-screen display, based on the target calories and the third value corresponding to the difference between the first value and the second value.

9. The electronic device of claim 1, further comprising an instruction which causes the processor to:
identify a target calorie intake amount corresponding to the target weight of the user; and
configure the first value, based on the target calorie intake amount.

10. An operation method of an electronic device, comprising:
acquiring data related to motion of a user carrying the electronic device;
determining a duration and a type of the motion, based at least partially on the acquired data;
acquiring a first value related to a food intake of the user;
determining a second value indicating an amount of calories burned by the user, based at least partially on the duration and the type of the motion and a weighted value related to the type of the motion; and
providing, based at least partially on the first value and the second value, a graphical user interface to a touch-screen display electrically connected to the electronic device.

11. The operation method of claim 10, wherein the providing of the graphical interface comprises providing the graphical user interface to the touch-screen display, based at least partially on a third value corresponding to a difference between the first value and the second value.

12. The operation method of claim 10, wherein the graphical user interface provides multiple ranges and indicates that the third value belongs to one of the multiple ranges.

13. The operation method of claim 10, wherein the determining of the second value comprises determining the second value, based at least partially on a profile of the user or a basal metabolic rate of the user.

14. The operation method of claim 10, further comprising:
determining a weight management mode, based on at least one of the profile of the user, a use history of the electronic device, or an input of the user;
configure target calories, based on a target weight of the user; and
providing the graphical user interface to the touch-screen display, based on the target calories and the third value corresponding to the difference between the first value and the second value.

15. The operation method of claim 10, wherein the acquiring of the first value comprises:
identifying a target calorie intake amount corresponding to the target weight of the user; and
configuring the first value, based on the target calorie intake amount.
